(19) United States
(12) Patent Application Publication
(10) Pub. No.: US 2022/0265312 A1
(43) Pub. Date: Aug. 25, 2022

(54) DEVICES FOR SAFE AND RELIABLE ACCESS TO SUB ARACHNOID AND SUBDURAL SPACE

(71) Applicants: SHPIGELMACHER Michael, Los Angeles, CA (US); BIONAUT LABS LTD., Herzliya (IL)

(72) Inventors: Michael SHPIGELMACHER, Los Angeles, CA (US); Michael KARDOSH, Kiryat Ono (IL); Be'eri Berl KATZNELSON, Kiryat Tivon (IL); Eran OREN, Tel Aviv (IL); Alex KISELYOV, San Diego, CA (US); Florent CROS, Los Angeles, CA (US); Suehyun CHO, Los Angeles, CA (US); Darrell HARRINGTON, Canoga Park, CA (US); Olin PALMER, Redwood City, CA (US)

(73) Assignee: BIONAUT LABS LTD., Herzliya (IL)

(21) Appl. No.: 17/628,089

(22) PCT Filed: Jul. 28, 2020
(86) PCT No.: PCT/US20/43859

§ 371 Date: Jan. 18, 2022

Related U.S. Application Data
(60) Provisional application No. 62/879,846, filed on Jul. 29, 2019.
(60) Provisional application No. 63/019,574, filed on May 4, 2020.

(51) Int. Cl. A61B 17/34; A61B 5/06

(52) U.S. Cl. CPC A61B 17/3401; A61B 5/062; A61B 2017/00876

(57) ABSTRACT

A method and apparatus for safely and reliably accessing a subarachnoid space (SAS) or other target area of a patient is provided in which a tube having at least one lumen with a tissue puncture device therein is inserted into the patient near the tissue. The lumen has a suction port at one end adapted to suction the dura/arachnoid tissue away from the pia mater so that the tissue can be punctured to access the SAS space. The movement of the needle in the lumen is controlled by controlling a magnetic field. Tissue puncture devices for use alone or in connection with the apparatus are also provided.

### FIELD OF THE INVENTION

The invention relates to apparatuses, devices, and methods for achieving safe and reliable access to anatomical spaces, such as subarachnoid and subdural spaces, in a patient.

### BACKGROUND

The spinal subarachnoid space (SAS) is the space between the arachnoid mater and pia mater in the spine and is continuous with the intracranial SAS. Spinal SAS communicates with the intracranial subarachnoid space via the foramen magnum and ends at the level of the S2 vertebra. It is a relatively large space, containing approximately half of the total volume of cerebrospinal fluid (CSF, 75 mLs out of 150 mLs). As the spinal cord ends at the level of L2, the subarachnoid space distal to this forms the lumbar cistern and is a preferred space to access the CSF via a lumbar puncture. The procedure is used in the clinical practice to analyze CSF, treat a variety of conditions including spinal pain or conduct diagnostics as exemplified by myelography.

Whereas this approach is well validated through decades of medical practice, there is a potential for transient or permanent spinal and/or more expanded trauma that includes paraplegia or even death. The ways in which nerve damage is caused include: a) direct injury caused by the needle or the catheter, b) hematoma, c) infection, d) disrupted hemodynamics, and e) misplacing the needle or the catheter. In addition, numerous cases of operational risk associated with the lumbar puncture have been reported. Of these, the actual dynamics of the procedure including the angle, speed, depth, stability of the needle/catheter insertion, placement and/or retraction is very operator-dependent.

Moreover, evidence suggests that spinal cord injuries due to the needle or catheter mediated procedures (ex., anesthesia, spinal root blocks) are rather common in the clinical practice. An illustrative example is the injury to a spinal nerve root by inaccurate and/or incautious needling during spinal anesthesia.

Postdural puncture headache (PDPH) incidence and severity are commonly assigned to size and nature of the dural hole produced during major neuraxial blockade or diagnostic dural puncture. Needle orientation in relation to the direction of dural fibers was thought to be of importance because of the propensity for horizontal bevel placement to cause cutting rather than splitting of the dural fibers. The utility of small 27G to 29G needles showed neither needle tip characteristics nor needle orientation had a substantial bearing on the damage to dural fibers in the dural lesion. However, the characteristic and size of the hole in the arachnoid was found to be critical. It has been noted that dural fibers tend to have enough “memory” to close back the hole created by a spinal needle, whereas arachnoid has diminished capacity to do so.

Occasionally, the needle may unintentionally enter the intrathecal space during lumbar interlaminar epidural steroid injections (LESIs)—one of the most commonly performed medical procedures in the United States. Ordinarily, this merely constitutes a minor complication or even a desired placement (in the case of some diagnostic procedures). However, some patients have a rare condition where the spinal cord terminates below the L2 vertebral level (tethered cord). In such cases, injections administered at the lumbar level may potentially result in spinal cord damage and irreversible paraplegia if the physician performing the intervention does not recognize the intramedullary position of the needle.

The pediatric population may pose specific challenges for proper needle positioning to avoid spinal trauma as the distance from the dura to spinal cord is not uniform at different vertebral levels. The dura to spinal cord distance may be a critical factor in avoiding the potential for neurological injury caused by needle trauma after a dural puncture. In children, the risk of spinal cord damage resulting from accidental epidural needle advancement may be greater in the lumbar region due to a more dorsal location of the spinal cord in the vertebral canal compared to the thoracic region.

Many physicians may consider lumbar injections “safe” because the spinal cord usually terminates at or above the L2 vertebral level. However, complacency stemming from this false impression of safety contributes to nonadherence to practice guidelines, which may lead to catastrophic neurological complications. There have been reports of paraplegia resulting from contrast medium injection into the spinal cord during a myelography study performed below the L2 vertebral level.

Cervical transforaminal epidural steroid injection (TFESI) under the guidance of computed tomography (CT) can offer great anatomical resolution and precise needle placement in the axial plane. However, some complications, including blood pressure surge, allergic reactions, vasovagal syncope, and cerebral infarct, have been reported after CT-guided cervical TFESI.

Despite the availability of specialized needles mediating access to the spine, these known needles carry with them certain risks and disadvantages. For example, recent studies suggest that both the Tuohy and Quincke needles may be more likely to cause trauma to the spinal compartment (ex., tibial nerve) than either the short-beveled or Whitacre needles.

Thus, there remains a need in the art for safer and more reliable configurations of tissue puncture devices, such as needles, for accessing anatomical spaces while overcoming the above-noted and other shortcomings of previously known devices and their uses.

### SUMMARY

It is desired to improve both safety and reliability of medical tissue puncture protocols. In particular, it is desired to improve both safety and reliability of a lumbar puncture protocol to access SAS and to reduce risks associated with the human factor. Devices described herein achieve these and other objectives, including real-time, objective detection of the SAS space; improved access success rate; reduced needle placement complications; and less time needed per procedure. Whereas the use of various needles and other tissue puncture devices known in the art has the potential of damaging Pia mater and/or various arteries, or not successfully passing into the SAS, embodiments of the present invention provide devices that achieve safe access to the SAS region.

Thus, in one aspect, the invention is an apparatus for providing surgical access to a sub arachnoid space bounded by a dura/arachnoid tissue layer and pia mater, wherein the apparatus comprises: a working tube having at least one lumen; a suction port at one end of the lumen configured to suction the arachnoid tissue layer positioned in front of the suction port; and a tissue puncture device susceptible to a magnetic field within the lumen configured to puncture the arachnoid tissue layer at the one end of the lumen to provide access to the subarachnoid space; and a magnetic field modulator adapted to move the tissue puncture device by controlling a magnetic field.

A method for accessing a subarachnoid space of a patient according to embodiments of the invention comprises inserting a tube having at least one lumen into the patient near the arachnoid tissue, said lumen having a suction port at one end adapted to suction the dura/arachnoid tissue away from the pia mater; moving a tissue puncture device susceptible to a magnetic field within the lumen by modulating the magnetic field; and puncturing the dura/arachnoid tissue with the tissue puncturing device.

In an embodiment, provided herein is a tissue puncturing device for accessing an anatomical space in a patient, said device comprising a main body extending along a longitudinal axis, said body comprising a distal end and a proximal end, said distal end comprising a tip, wherein the tip comprises a shoulder region characterized by an abrupt narrowing of diameter which terminates in a sharp point at its most distal end.

In an embodiment, provided herein is a tissue puncturing device for accessing an anatomical space in a patient, said device comprising a main body extending along a longitudinal axis, said body comprising a hollow core, a distal end, and a proximal end, said distal end comprising a tip, wherein the tip comprises a shoulder region characterized by a narrowing of diameter which terminates in a point at its most distal end, and wherein the hollow core is configured to permit passage of a fluid into or out of the distal end of the tip.

In an embodiment, provided herein is a tissue puncturing device for accessing an anatomical space in a patient, said device comprising a main body extending along a longitudinal axis, said body comprising a hollow core, a distal end, and a proximal end, said distal end comprising a tip, wherein the tip comprises a shoulder region characterized by a narrowing of diameter which terminates in an ogive shaped distal region that is closed at its most distal end, and wherein the ogive shaped distal region comprises a side port configured to permit a fluid or an object to enter or exit therethrough.

In an embodiment, provided herein is a tissue puncturing device for accessing an anatomical space in a patient, said device comprising a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end shaped to terminate with a sharp spear structure and wherein the distal region comprises a side port configured to permit a fluid or an object to enter or exit therethrough.

In an embodiment, provided herein is a tissue puncturing device for accessing an anatomical space in a patient, said device comprising a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising an apex that is flattened in one direction, and wherein the distal region comprises a side port configured to permit a fluid or an object to enter or exit therethrough.

In an embodiment, provided herein is a tissue puncturing device for accessing an anatomical space in a patient, said device comprising a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising two flat planar surfaces on each side, and wherein the distal region comprises a side port configured to permit a fluid or an object to enter or exit therethrough.

In an embodiment, the tissue puncturing device is a needle.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts the anatomical and physiological landmarks, including, e.g. subarachnoid space, of a spinal environment in which embodiments of the present invention can be employed.

FIG. 2 schematically depicts a needle inserted inside the subarachnoid space by means of a shaft around the needle in accordance with an embodiment of the invention.

FIG. 3 schematically depicts a stage in a method wherein the lumen approaches the subarachnoid space with a sensor inside, in accordance with an embodiment of the invention.

FIG. 4 schematically depicts a stage in the process where the lumen is near the subarachnoid space and suction is applied before puncture, to enlarge space for the needle to puncture.

FIG. 5 depicts a spinal cord with intercostal nerves.

FIG. 6 depicts a detail of a spinal cord and surrounding physiological landmarks.

FIGS. 7A-7E depict tissue puncture devices in accordance with various embodiments of the invention.

FIGS. 8A-8I depict tissue puncture devices in accordance with various embodiments of the invention.

FIGS. 9A-9B depict a use of a tissue puncture device in accordance with an embodiment of the invention.

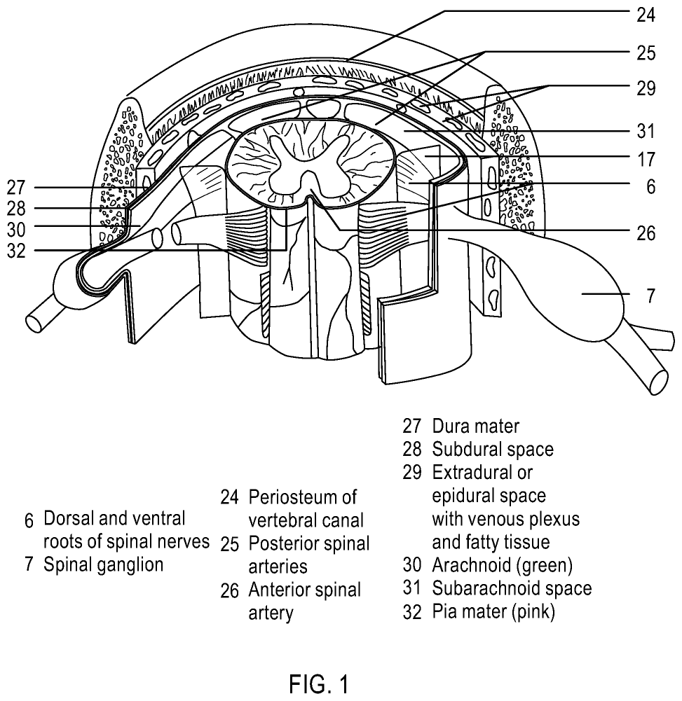

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Specific embodiments of the present invention are now described, including with reference to the figures, wherein like reference numbers indicate identical or functionally similar elements.

Unless otherwise defined herein, scientific and technical terms used in connection with this application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The terms “subject,” “individual,” and “patient” are used interchangeably herein. The term “subject” as used herein refers to human and non-human animals. The human can be any human of any age. In an embodiment, the human is an adult. In another embodiment, the human is a child.

Although the description of the invention is generally in the context of accessing subarachnoid and subdural spaces, the invention may also be used in any other body passageways or areas where it is deemed useful. For example, an anatomical space or locus that can be accessed by devices and apparatuses described herein, and/or for which controllability, safety, stability, and reliable access are desired, is a suitable target for use of the invention. There is no intention to be bound by any express or implied theory presented in the preceding technical field, background, brief summary or the following detailed description.

A device according to one embodiment of the invention uses a working channel/tube containing one or more lumens. In an embodiment, inside the lumen, the device may contain one or more of the following: (1) a pressure sensor; (2) an ultrasound transducer; (3) a suction port enabling suction of the tissue in front of the port; (4) an imaging port; (5) an electric sensor enabling measuring tissue impedance with at least one electrode on the tip of a needle in the channel/tube; (6) an optical, electric, piezoelectric, (electro)magnetic, RF or ultrasonic stimulator; and (7) a mechanical manipulator, such as tweezers/needle.

In an embodiment, the ultrasound transducer may be adapted for one or more of: imaging; speed assessment using Doppler; thickness assessment of the tissue layers; and assessing flexibility/rigidity of the layers in front of the tip using changes in SOS (speed of sound) in different media.

In an embodiment, the suction port may be operated so that the tissue layer in front of the port is pulled and the tissue is punctured in a more distant location from the nerve than the original arachnoid tissue position (because farther from the Pia is a safer location). The suction port may also be used in administering a treatment including but not limited to mechanical, thermal or alternative physical force field and/or specific therapeutic formulations, diagnostics, contrasting and/or other imaging agents. Therapeutic formulations may constitute a solution, suspension, gel/sol or alternative modalities.

The aforementioned components are expected to mediate identification, differentiation and proper positioning of potential treatment summarized above at/between the compartments and tissue(s) of interest including but not limited to spinal dura mater, arachnoid matter, pia matter based on specific (bio)physical parameters including appearance, conductivity/resistance, mechanical properties, flow and other criteria.

In a proposed protocol of one or more of the embodiments described herein, once the arachnoid layer is identified and contacted, the needle is inserted. The device may contain an insertion controller to make sure the needle does not exceed the desired depth. Furthermore, the device may be automated to include an inserting mechanism that inserts the needle in a controlled manner to prevent injury.

In another embodiment, described herein is a puncture device for safe and reliable access to areas of tissue or spaces between tissue, as desired, such as the subarachnoid space. In an embodiment, the subarachnoid space is intracranial subarachnoid space. In an embodiment, the subarachnoid space is spinal subarachnoid space. In an embodiment, the spinal subarachnoid space is lumbar subarachnoid space.

In an embodiment, the puncture device is a needle. In embodiments, devices are designed and constructed based on optimized topology of a penetrating tip aimed at better control, to minimize or eliminate spinal or other trauma.

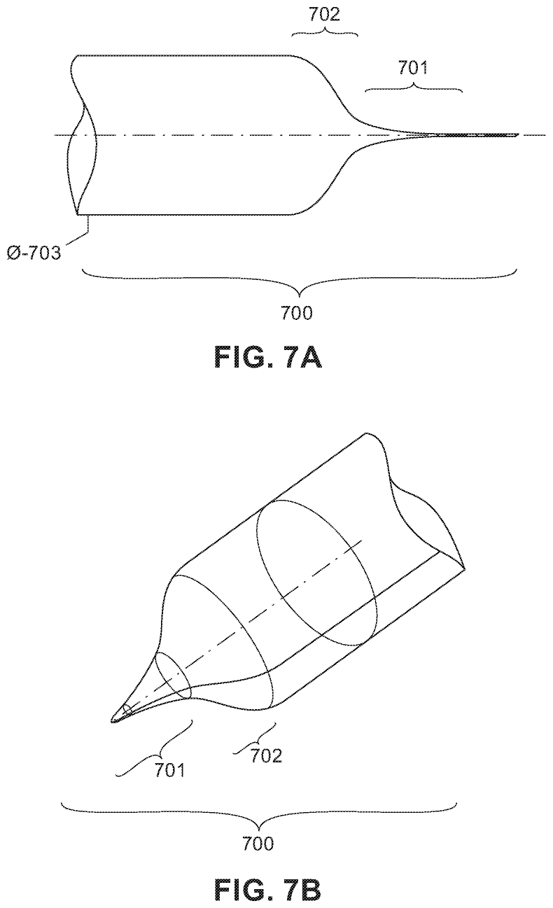

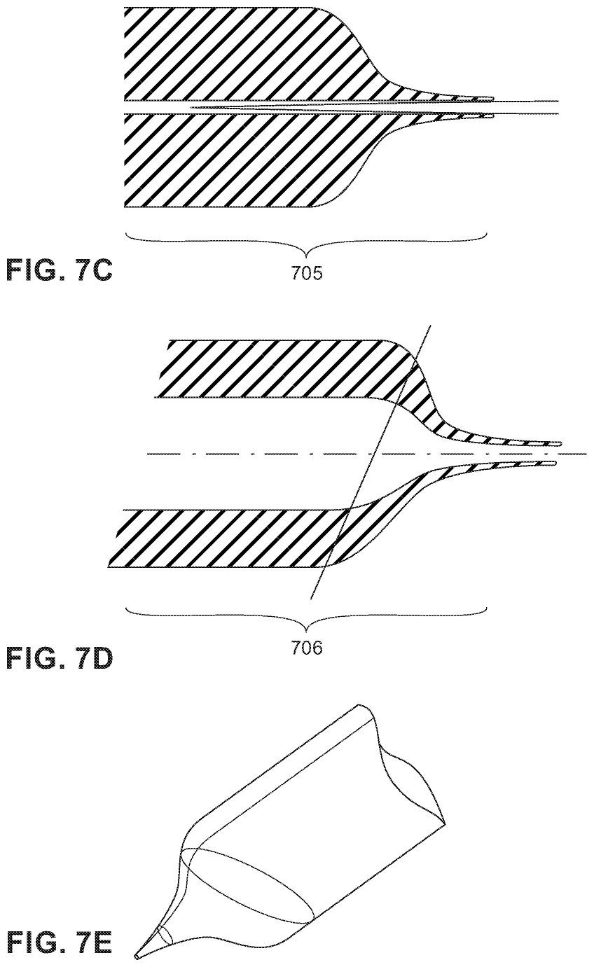

In the embodiment illustrated in FIGS. 7A and 7B, the tissue puncture device comprises a solid tip. In the illustrated embodiment, the device is a needle [700], although in various alternate embodiments, the device is not limited to a needle and may be any suitable device for performing the functions and achieving the desired effects described herein. In an embodiment, wherein a device described herein is or comprises a needle, the needle can be any suitable needle, such as, without limitation a spinal or epidural needle. In the illustrated embodiment, the device [700] comprises a profile that varies rapidly at the distal end. In an embodiment, the most distal section of the tip is shaped like a cone. In an embodiment, the cone is affixed to a cylinder whose outside diameter remains constant or nearly constant along an appropriate specified distance, after which the outside diameter increases over a certain distance and reaches a local maxima. In an embodiment, the maxima can remain constant afterward for an appropriate specified distance. In one embodiment, the tip is a solid cylinder with average diameter [703]. In an embodiment, the most distal part of the tip [701] is sharp, i.e. with very small radius of curvature at its apex, an average diameter substantially smaller than that of the more proximal portion [703]. In a representative example, the diameter of the distal region [701] is 75 μm or less. In an embodiment, the outer diameter of the most distal region can be between about 10 μm and about 1000 μm. In an embodiment, the outer diameter of the most distal region can be between about 10 μm and about 100 μm. In an embodiment, the outer diameter of the most distal region can be between about 100 μm and about 500 μm. In an embodiment, the outer diameter of the most distal region can be between about 50 μm and about 100 μm. In an embodiment, the outer diameter of the most distal region can be between about 50 μm and about 80 μm. In an embodiment, the outer diameter of the most distal region can be between about 10 μm and about 50 μm.

In an embodiment, the total length of the most distal region, which includes the cone and a cylinder with constant or nearly constant outer diameter can be between about 50 μm and about 2000 μm. In an embodiment, the total length of the most distal region, which includes the cone and a cylinder with constant or nearly constant outer diameter can be between about 50 μm and about 1000 μm. In an embodiment, the total length of the most distal region, which includes the cone and a cylinder with constant or nearly constant outer diameter can be between about 1000 μm and about 2000 μm. In an embodiment, the total length of the most distal region, which includes the cone and a cylinder with constant or nearly constant outer diameter can be between about 50 μm and about 100 μm. In an embodiment, the total length of the most distal region, which includes the cone and a cylinder with constant or nearly constant outer diameter can be between about 100 μm and about 500 μm. In an embodiment, the total length of the most distal region, which includes the cone and a cylinder with constant or nearly constant outer diameter can be between about 500 μm and about 750 μm.

The distal region's [701] overall length can be chosen to closely match the thickness of the tissue or layer to be punctured. In one embodiment, the length of the distal region [701] is equal to or slightly longer than the medium to be punctured. In another embodiment, the length is several times the length of the medium to be punctured. Next to the distal region [701], traveling toward the proximal end of the device [700] is a shoulder region [702] characterized by a rapid change in diameter over a relatively short distance. The shoulder region's [702] profile can be tailored to the needs of a specific application, as understood and determined by one of ordinary skill in the art. In use, such as when attempting to advance a tip and traverse the Dura, a layer of tissue around the spinal cord, the shoulder region [702] can be short and the transition in diameter as abrupt as possible to achieve a desirable rapid increase in contact force between the needle and the Dura tissue, once the needle tip advances to a desired distance beyond the Dura.

In the embodiment illustrated in FIG. 7C, the tip [705] is hollow and allows passage of homogeneous or heterogeneous fluids therethrough. Such fluids can be any suitable fluids, such as, without limitation, solutions, suspensions, emulsions, gels, sols or combinations thereof.

In the embodiment illustrated in FIGS. 7D and 7E, [706], the length of the distal region [701] varies along the circumference. This configuration can be adapted to a situation in which, for example, the tip is used to penetrate a medium at an angle that is not immediately perpendicular to the upper surface of the medium.

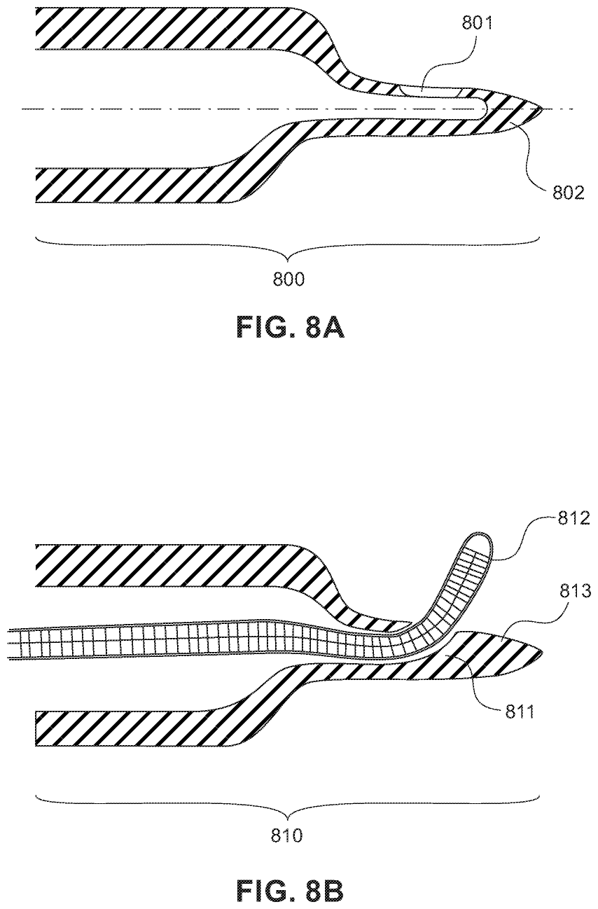

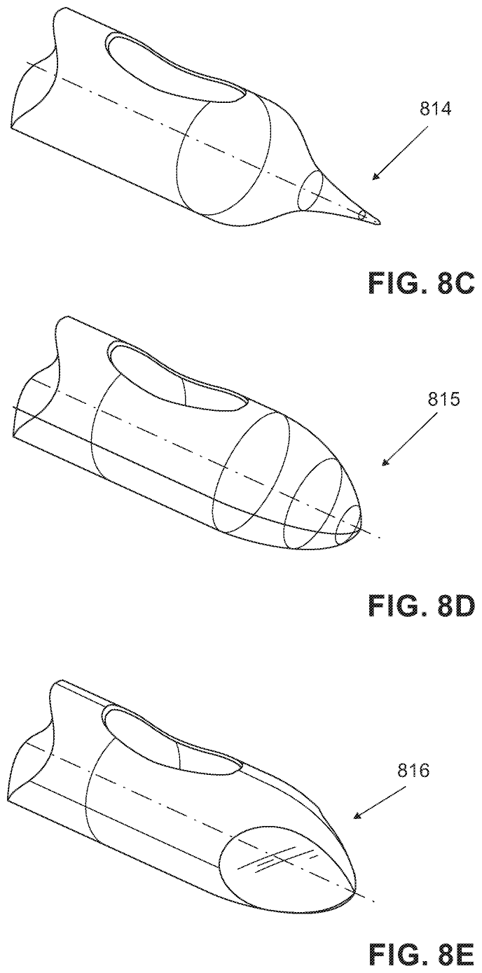

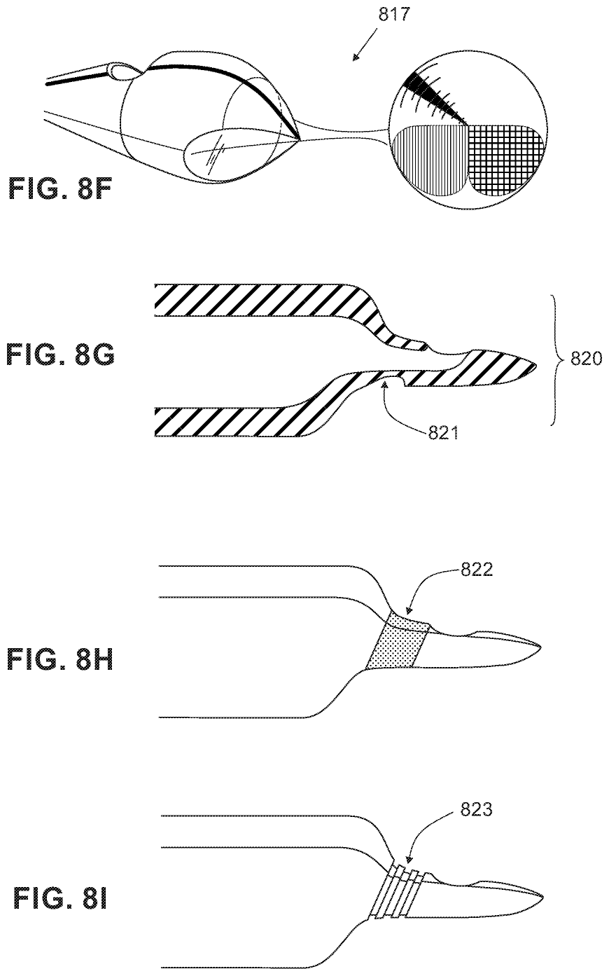

In the embodiment illustrated in FIG. 8A the tip [800], exemplified here by a needle, is hollow. The most distal part of the needle [802] is closed and shaped like an ogive or a bullet. A side port [801] is created to allow fluids to either escape from or be sucked in.

In the embodiment illustrated in FIG. 8B, [810], the needle is hollow. The most distal part of the needle is closed. The hollowed-out region near the tip of the needle [813] is shaped to allow for an object, a series thereof, a line, a tether [812] or any other suitable object to travel in and out of the needle smoothly and to come out at a predefined angle. An area [811], internal to the hollowed-out channel inside the needle is shaped with a slope to ease the passage of the object(s) [812] and to deflect the traversing media, object or line from the adjacent tissue. This tissue can be, for example, without limitation, pia matter/spinal cord or any other tissue dependent on the environment in which the device is appropriately used.

In an embodiment, the tip [813] is shaped to terminate with a sharper spear structure, as shown [814] in FIG. 8C. In another embodiment, the tip [813] resembles that depicted [815] in FIG. 8D, where the apex of the needle is flattened in one direction. This topology can be useful, for example, when puncturing a medium that has a heterogeneous structure, such as, without limitation, fibrous, perfused locus, tissue, compartment and/or organ. Specifically, this topology renders the act of penetration easier if the flattened part is oriented in a fashion that separates the fibers rather than cuts, slices or severs them.

In the embodiment illustrated in FIG. 8E, [816], two flat planes are ground or otherwise formed on each side of the needle ogive.

In the embodiment illustrated in FIG. 8F, [817], the two planes meet to create a sharp edge to ease the cutting of the medium in one direction. This could be useful when the needle is advanced into the medium of interest, such as for example, without limitation, to a specific locus, tissue, compartment, or organ, at an oblique angle.

In the embodiment illustrated in FIG. 8G, [820], a depression [821] along the external wall of the needle provides a means to latch and hold on to the medium during the puncturing phase, thus providing controlled travel distance within the compartment of interest and limiting potential for trauma. Alternatively, the surface topology of the outside wall can be designed to provide grip or friction [822] between the needle and the medium to be punctured, as seen in the embodiment illustrated in FIG. 8H. This surface topology modification could be asymmetric along the needle axis (i.e. a “barbed” needle), so as to provide a significant increase in friction during needle retraction without significantly increasing friction during needle advancement.

As shown in the embodiment illustrated in FIG. 8I, one or more groove(s) [823] can be machined or otherwise formed on the outside wall of the needle to enhance the friction between the needle and the medium to puncture.

Referring again to FIG. 8G, the embodiment illustrated therein [820] can be used to puncture, e.g., the Dura and advance a guide wire.

In an embodiment, the device is used to puncture the Dura of the spinal cord. Due to the sharp apex, the dura can be punctured with ease wherein the over oblong or ogive shape of the apex yields minimal laceration of nerves. Moreover, the shoulder [802] provided by the enlargement of the needle provides added safety as the sharpest part of the needle cannot penetrate too deep into the spine. The depression [821] allows the operator to tug back on the needle and to retract the needle tip away from the center of the spinal cord while ensuring that the side port opening [801] is still residing under the Dura. The hollowed-out part of the needle and the slope leading yields an option of traversing a guide wire that can be safely introduced under the Dura, away from other parts of the cord, including nerves and pia mater.

In an embodiment, it is envisioned that the described and illustrated solid tip topology featuring, for example, i) short sharp tip followed by ii) rapidly proximally increasing body size featuring holding notch(es) and iii) overall solid or hollow structure to be of general use for controlled penetration and delivery of therapeutic or diagnostic payload to loci, tissue(s), compartment(s), organ(s) adjacent to anatomically and physiologically sensitive structures as represented by, but not limited to a spinal cord, specific brain circuitry, neuronal plexuses proximal to major vascular bed, or other suitable target or region.

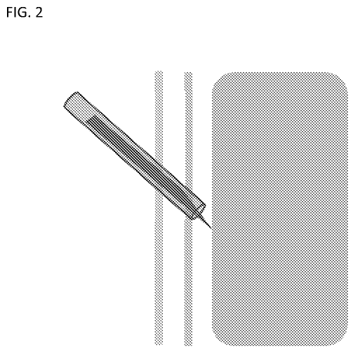

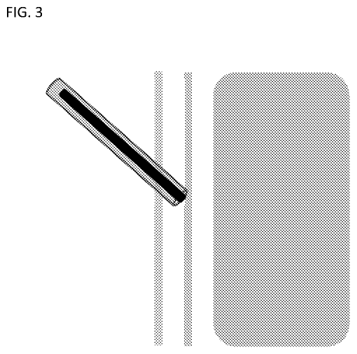

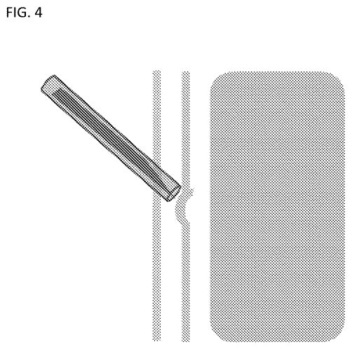

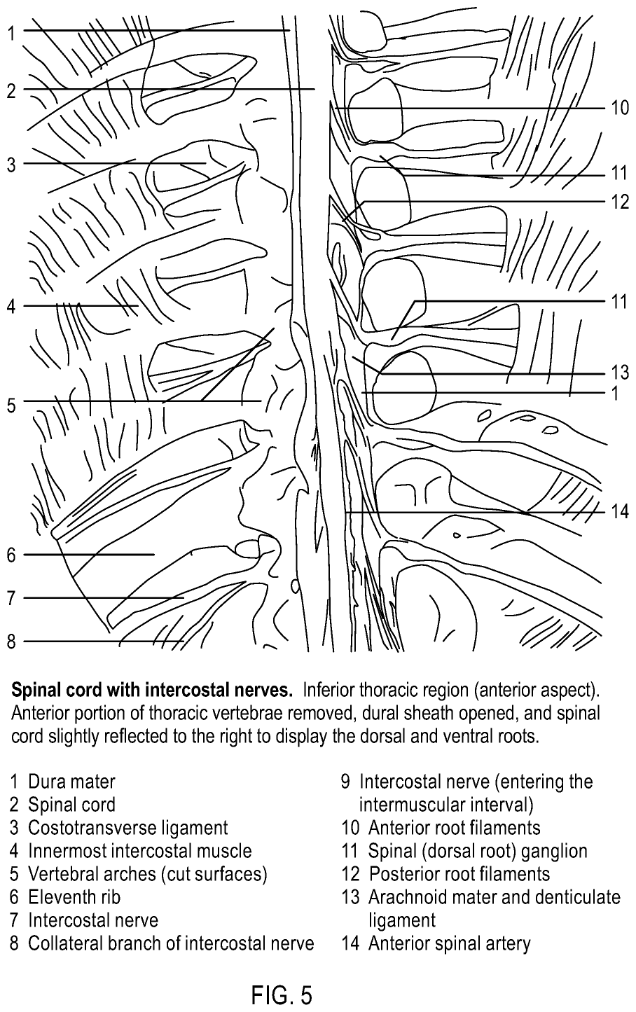

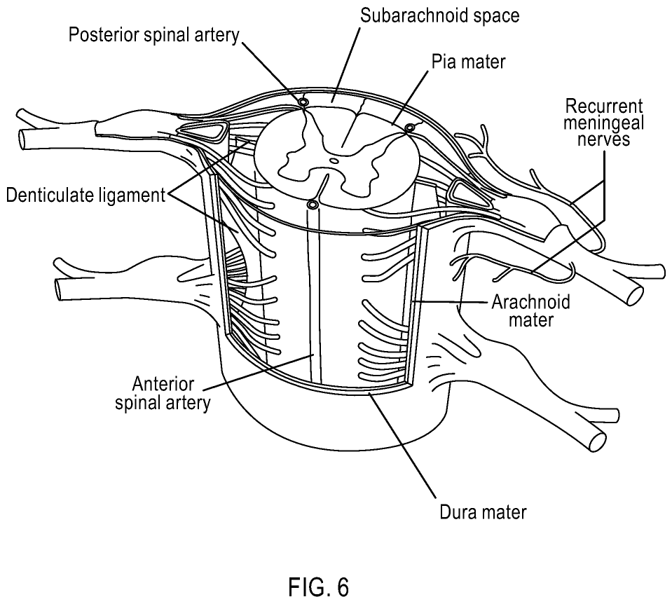

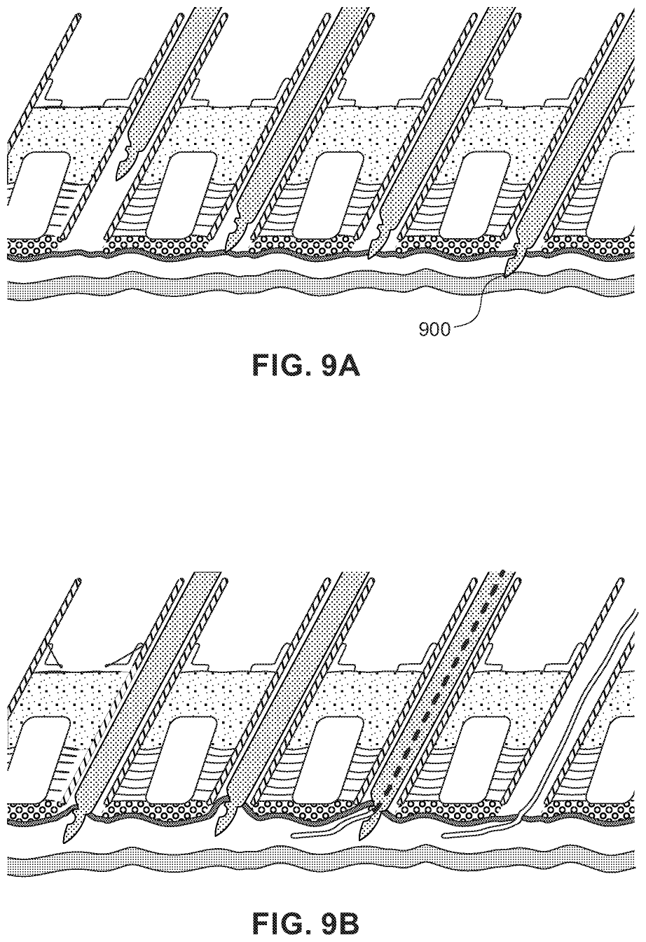

The embodiments of devices described herein, including in FIGS. 7-9, can be employed alone or in connection with the apparatus of FIGS. 2-4, such as, for example as the needle described therein or with any other suitable apparatus or system for puncturing tissue, as appropriate.

Suitable materials that may be employed in construction of the devices described herein include, without limitation, stainless steel, titanium, gold, polyether ethyl ketone (PEEK), or any other material or combination of materials appropriate to achieve the desired properties and results as described herein.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An apparatus for providing surgical access to a sub arachnoid space bounded by a dura/arachnoid tissue layer and pia mater, comprising:
a working tube having at least one lumen;
a suction port at one end of the lumen configured to suction the arachnoid tissue layer positioned in front of the suction port; and
a tissue puncture device susceptible to a magnetic field within the lumen configured to puncture the arachnoid tissue layer at the one end of the lumen to provide access to the subarachnoid space; and
a magnetic field modulator adapted to move the tissue puncture device by controlling a magnetic field.

2. The apparatus according to claim 1, further comprising an imaging port within the working tube.

3. The apparatus according to claim 2, wherein the imaging port houses an ultrasound transducer for ultrasound imaging.

4. The apparatus according to claim 1, further comprising an ultrasound transducer within the lumen configured to assess the speed of the tissue puncture device using Doppler effect.

5. The apparatus according to claim 1, further comprising an ultrasound transducer within the lumen configured to assess the flexibility of tissue in front of the suction port in response to a measurement of the speed of sound through the arachnoid tissue and surrounding tissue.

6. The apparatus according to claim 1, further comprising an electrical sensor measuring electrical impedance or related parameter of the arachnoid tissue with at least one electrode positioned on the tissue puncture device.

7. The apparatus according to claim 1, wherein the suction port is adapted to suction the arachnoid tissue layer away from the pia mater.

8. The apparatus according to claim 1, further comprising a mechanical manipulator.

9. The apparatus according to claim 1, further comprising an optical, electrical, magnetic, RF or ultrasonic stimulator.

10. The apparatus according to claim 1, further comprising a pressure sensor measuring pressure inside the lumen.

11. The apparatus according to claim 1, further comprising a treatment modality operating in the sub arachnoid space.

12. The apparatus according to claim 11, wherein the treatment modality comprises administering a composition to the subarachnoid space.

13. The apparatus according to claim 1, wherein the apparatus is equipped with LEDs or relevant optical array at the tip, sides or alternative relevant placement to mediate optical imaging, tracking and/or data collection.

14. The apparatus of any of claims 1-13, wherein the tissue puncture device comprises a needle.

15. The apparatus of claim 14, wherein the needle is an epidural or spinal needle.

16. A method for accessing a subarachnoid space of a patient, said space being bounded by dura/arachnoid tissue and pia mater, comprising:
inserting a tube having at least one lumen into the patient near the arachnoid tissue, said lumen having a suction port at one end adapted to suction the dura/arachnoid tissue away from the pia mater;
moving a tissue puncture device susceptible to a magnetic field within the lumen by modulating the magnetic field;
puncturing the dura/arachnoid tissue with the tissue puncture device.

17. The method according to claim 16, further comprising administering a composition to the subarachnoid space.

18. The method according to claim 16, further comprising imaging an area proximate the subarachnoid tissue during a procedure.

19. The method according to claim 16, further comprising measuring position and speed of the tissue puncture device throughout a procedure.

20. The method according to claim 16, further comprising measuring the flexibility of dura/arachnoid tissue positioned in front of the suction port.

21. The method according to claim 16, further comprising measuring electrical impedance of the arachnoid tissue with at least one electrode positioned on the tissue puncture device.

22. The method of any of claims 16-21, wherein the tissue puncture device comprises a needle.

23. The method of claim 22, wherein the needle is an epidural or spinal needle.

24. The method of any of claims 16-23, wherein the subarachnoid space comprises spinal subarachnoid space.

25. The method of claim 24, wherein the spinal subarachnoid space comprises lumbar subarachnoid space.

26. The apparatus of any of claims 1-15, wherein the tissue puncture device comprises a main body extending along a longitudinal axis, said body comprising a distal end and a proximal end, said distal end comprising a tip, wherein the tip comprises a shoulder region characterized by an abrupt narrowing of diameter which terminates in a sharp point at its most distal end.

27. The apparatus of any of claims 1-15, wherein the tissue puncture device comprises a main body extending along a longitudinal axis, said body comprising a hollow core, a distal end, and a proximal end, said distal end comprising a tip, wherein the tip comprises a shoulder region characterized by a narrowing of diameter which terminates in a point at its most distal end, and wherein the hollow core is configured to permit passage of a fluid into or out of the distal end of the tip.

28. The apparatus of claim 27, wherein the shoulder region has a length that varies along its circumference.

29. The apparatus of any of claims 1-15, wherein the tissue puncture device comprises a main body extending along a longitudinal axis, said body comprising a hollow core, a distal end, and a proximal end, said distal end comprising a tip, wherein the tip comprises a shoulder region characterized by a narrowing of diameter which terminates in an ogive shaped distal region that is closed at its most distal end, and wherein the ogive shaped distal region comprises a side port configured to permit a fluid or an object to enter or exit therethrough.

30. The apparatus of claim 29, wherein the shoulder region has a length that varies along its circumference.

31. The apparatus of any of claims 1-15, wherein the tissue puncture device comprises a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end shaped to terminate with a sharp spear structure and wherein the distal region comprises a side port configured to permit a fluid or an object to enter or exit therethrough.

32. The apparatus of any of claims 1-15, wherein the tissue puncture device comprises a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising an apex that is flattened in one direction, and wherein the distal region comprises a side port configured to permit a fluid or an object to enter or exit therethrough.

33. The apparatus of any of claims 1-15, wherein the tissue puncture device comprises a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising two flat planar surfaces on each side, and wherein the distal region comprises a side port configured to permit a fluid or an object to enter or exit therethrough.

34. The apparatus of claim 33, wherein the two planar surfaces meet to create a sharp edge.

35. The apparatus of any of claims 26-34, further comprising a depression disposed along the exterior of the device configured to hold the device to the medium during puncturing.

36. The apparatus of any of claims 26-34, further comprising a means for providing a grip or friction between the device and the medium to be punctured.

37. The apparatus of any of claims 26-34, further comprising at least one annular groove on the device, configured to enhance friction between the device and the medium during puncturing.

38. The method of any of claims 16-25, wherein the tissue puncture device comprises a main body extending along a longitudinal axis, said body comprising a distal end and a proximal end, said distal end comprising a tip, wherein the tip comprises a shoulder region characterized by an abrupt narrowing of diameter which terminates in a sharp point at its most distal end.

39. The method of any of claims 16-25, wherein the tissue puncture device comprises a main body extending along a longitudinal axis, said body comprising a hollow core, a distal end, and a proximal end, said distal end comprising a tip, wherein the tip comprises a shoulder region characterized by a narrowing of diameter which terminates in a point at its most distal end, and wherein the hollow core is configured to permit passage of a fluid into or out of the distal end of the tip.

40. The method of claim 39, wherein the shoulder region has a length that varies along its circumference.

41. The method of any of claims 16-25, wherein the tissue puncture device comprises a main body extending along a longitudinal axis, said body comprising a hollow core, a distal end, and a proximal end, said distal end comprising a tip, wherein the tip comprises a shoulder region characterized by a narrowing of diameter which terminates in an ogive shaped distal region that is closed at its most distal end, and wherein the ogive shaped distal region comprises a side port configured to permit a fluid or an object to enter or exit therethrough.

42. The method of claim 41, wherein the shoulder region has a length that varies along its circumference.

43. The method of any of claims 16-25, wherein the tissue puncture device comprises a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end shaped to terminate with a sharp spear structure and wherein the distal region comprises a side port configured to permit a fluid or an object to enter or exit therethrough.

44. The method of any of claims 16-25, wherein the tissue puncture device comprises a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising an apex that is flattened in one direction, and wherein the distal region comprises a side port configured to permit a fluid or an object to enter or exit therethrough.

45. The method of any of claims 16-25, wherein the tissue puncture device comprises a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising two flat planar surfaces on each side, and wherein the distal region comprises a side port configured to permit a fluid or an object to enter or exit therethrough.

46. The method of claim 45, wherein the two planar surfaces meet to create a sharp edge.

47. The method of any of claims 16-25, further comprising a depression disposed along the exterior of the device configured to hold the device to the medium during puncturing.

48. The method of any of claims 16-25, further comprising a means for providing a grip or friction between the device and the medium to be punctured.

49. The method of any of claims 16-25, further comprising at least one annular groove on the device, configured to enhance friction between the device and the medium during puncturing.

50. A tissue puncturing device for accessing an anatomical space in a patient, said device comprising a main body extending along a longitudinal axis, said body comprising a distal end and a proximal end, said distal end comprising a tip, wherein the tip comprises a shoulder region characterized by an abrupt narrowing of diameter which terminates in a sharp point at its most distal end.

51. A tissue puncturing device for accessing an anatomical space in a patient, said device comprising a main body extending along a longitudinal axis, said body comprising a hollow core, a distal end, and a proximal end, said distal end comprising a tip, wherein the tip comprises a shoulder region characterized by a narrowing of diameter which terminates in a point at its most distal end, and wherein the hollow core is configured to permit passage of a fluid into or out of the distal end of the tip.

52. The device of claim 51, wherein the shoulder region has a length that varies along its circumference.

53. A tissue puncturing device for accessing an anatomical space in a patient, said device comprising a main body extending along a longitudinal axis, said body comprising a hollow core, a distal end, and a proximal end, said distal end comprising a tip, wherein the tip comprises a shoulder region characterized by a narrowing of diameter which terminates in an ogive shaped distal region that is closed at its most distal end, and wherein the ogive shaped distal region comprises a side port configured to permit a fluid or an object to enter or exit therethrough.

54. The device of claim 53, wherein the shoulder region has a length that varies along its circumference.

55. A tissue puncturing device for accessing an anatomical space in a patient, said device comprising a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end shaped to terminate with a sharp spear structure and wherein the distal region comprises a side port configured to permit a fluid or an object to enter or exit therethrough.

56. A tissue puncturing device for accessing an anatomical space in a patient, said device comprising a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising an apex that is flattened in one direction, and wherein the distal region comprises a side port configured to permit a fluid or an object to enter or exit therethrough.

57. A tissue puncturing device for accessing an anatomical space in a patient, said device comprising a main body extending along a longitudinal axis, said body comprising a hollow core, a distal end, and a proximal end, said distal end comprising a tip, wherein the tip optionally comprises a shoulder region characterized by a narrowing of diameter which terminates in a distal region that is closed at its most distal end, said most distal end comprising two flat planar surfaces on each side, and wherein the distal region comprises a side port configured to permit a fluid or an object to enter or exit therethrough.

58. The device of claim 57, wherein the two planar surfaces meet to create a sharp edge.

59. The device of any of claims 50-58, further comprising a depression disposed along the exterior of the device configured to hold the device to the medium during puncturing.

60. The device of any of claims 50-58, further comprising a means for providing a grip or friction between the device and the medium to be punctured.

61. The device of any of claims 50-58, further comprising at least one annular groove on the device, configured to enhance friction between the device and the medium during puncturing.
